(19) United States
(12) Patent Application Publication
(10) Pub. No.: US 2023/0414611 A1
(43) Pub. Date: Dec. 28, 2023

(54) REFINED USES OF GABA A RECEPTOR MODULATORS IN TREATMENT OF FRAGILE X SYNDROME

(71) Applicant: CHILDREN'S HOSPITAL MEDICAL CENTER, Cincinnati, OH (US)

(72) Inventors: Craig A. ERICKSON, Cincinnati, OH (US); Christina GROSS, Cincinnati, OH (US); Ernest PEDAPATI, Cincinnati, OH (US); Lauren SCHMITT, Cincinnati, OH (US)

(73) Assignee: CHILDREN'S HOSPITAL MEDICAL CENTER, Cincinnati, OH (US)

(21) Appl. No.: 18/252,813

(22) PCT Filed: Nov. 12, 2021
(86) PCT No.: PCT/US2021/059192

§ 371 Date: May 12, 2023

Related U.S. Application Data
(60) Provisional application No. 63/125,323, filed on Dec. 14, 2020.
(60) Provisional application No. 63/113,207, filed on Nov. 13, 2020.

(51) Int. Cl. A61K 31/502; A61K 9/00; A61K 45/06; A61P 25/00; G01N 33/68; A61B 5/374

(52) U.S. Cl. CPC A61K 31/502; A61K 9/0053; A61K 45/06; A61P 25/00; G01N 33/6896; A61B 5/374; G01N 2800/28

(57) ABSTRACT

Identification of Fragile X syndrome (FXS) patients who are likely to respond to a treatment involving a GABA_{A} receptor modulator (e.g., a selective GABA_{A} receptor stimulator), for example, at a low dose, using peripheral Fragile X mental retardation protein (FMRP) as a biomarker, either taken alone or in combination with other biomarkers. Also provided here are methods for treating such FXS patients using the GABA_{A} receptor modulator (e.g., a selective GABA_{A} receptor stimulator at a low dose).

### CROSS REFERENCE TO RELATED APPLICATIONS

This application claims the benefit of the filing dates of U.S. Provisional Application No. 63/113,207, filed Nov. 13, 2020 and U.S. Provisional Application No. 63/125,323, filed Dec. 14, 2020, the entire contents of each of which are incorporated by reference herein.

### BACKGROUND OF THE INVENTION

Fragile X Syndrome (FXS) is a monogenetic syndrome caused by an expansion of CGG repeats in the fragile X mental retardation protein (FMR1) gene which results in the loss of the gene product, the Fragile X mental retardation protein (FMRP), and the leading cause of inherited intellectual disability. Individuals with FXS have low IQs, are developmentally delayed, have impairments in verbal and nonverbal communication (often meeting ASD criteria), and suffer from neuronal hyperexcitability that becomes manifest in hypersensitivity to sound and light and in epileptic seizures.

Individuals with FXS need lifelong care and cannot live independent lives, reducing life quality for affected individuals and their caregivers. There is a need to develop new therapies for the treatment of FXS.

### SUMMARY OF THE INVENTION

The present disclosure is based, at least in part, on the unexpected results obtained from a human clinical trial on FXS that patients (e.g., male patients) having undetectable peripheral FMRP showed responsiveness to low dose AZD7325 (a representative GABA_{A} modulator, also known as BEAR-101) treatment, while patients having detectable peripheral FMRP were found not responsive to the treatment. Further, it was found that, unexpectedly, gamma EEG values correlate with treatment efficacy of low dose AZD7325. Accordingly, the level of peripheral FMRP, either taken alone or in combination with gamma EEG values, can be used as a reliable biomarker for identifying FXS patients who are likely to respond to GABA_{A} modulator treatment (e.g., low dose GABA_{A} modulator treatment).

Accordingly, the present disclosure, in some aspects, features a method for identifying a Fragile X Syndrome (FXS) patient who is likely to respond to a treatment for FXS (FXS treatment), the method comprising: (a) providing a biological sample of a FXS patient; (b) detecting Fragile X mental retardation protein (FMRP) in the biological sample; and (c) identifying the FXS patient for the FXS treatment, when the FMRP is undetectable in the biological sample or has a level no greater than (e.g., lower than) a predetermined value. In some instances, the FXS treatment comprises a GABA_{A} receptor modulator, a muscle relaxer, a gene therapy comprising a vector for expressing a functional FMRP (e.g., a wild-type FMRP polypeptide), or a combination thereof. In some examples, the FXS treatment comprises the GABA_{A} receptor modulator (e.g., AZD7325). In some examples, the FXS patient is a male human patient.

Any of the methods disclosed herein may further comprise performing gamma electroencephalogram (EEG) on the FXS patient to measure electrical activity of the brain. Alternatively or in addition, the method may further comprise subjecting the FXS patient identified in any of the methods disclosed herein to an FXS treatment. For example, the method may further comprise administering to the FXS patient identified in step (c) an effective amount of a GABA_{A} modulator (e.g., a low dose of the GABA_{A} modulator). In some examples, the GABA_{A} modulator may be a selective GABA_{A} agonist. In specific examples, the FXS patient may be administered AZD7325 (e.g., at 5 mg twice per day). In some instances, the GABA_{A} modulator such as AZD7325 may be administered orally. Alternatively or in addition, the FXS treatment may comprise a muscle relaxer such as baclofen (e.g., racemic baclofen).

In other aspects, the present disclosure features a method for treating Fragile X syndrome (FXS), comprising subjecting an FXS patient (e.g., a male FXS patient) to a treatment for FXS (FXS treatment). The FXS patient has undetectable peripheral Fragile X mental retardation protein (FMRP) or a level of peripheral FMRP no greater than (e.g., lower than) a predetermined value. In some embodiments, the FXS treatment comprises an effective amount of a GABA_{A} modulator, for example, a selective GABA_{A} agonist; a muscle relaxer such as baclofen, or a vector for producing functional FMRP (gene therapy). In some examples, the FXS patient may be administered a low dose of the GABA_{A} modulator. Alternatively or in addition, the GABA_{A} modulator may be administered to the FXS patient orally.

In some examples, the FXS treatment comprises a GABA_{A} modulator, which can be AZD7325. In specific examples, the FXS treatment may comprise AZD7325 at 5 mg twice per day.

Any of the methods disclosed herein may further comprise performing gamma electroencephalogram (EEG) on the FXS patient before, during, and/or after the FXS treatment to monitor treatment efficacy.

In any of the methods disclosed herein, the biological sample for measurement of FMRP is a biofluid sample. Examples include a blood sample, a plasma sample, or a serum sample. In some examples, the biological sample is a dried blood spot sample. In some embodiments, the presence or level of FMRP in the biological sample may be measured by an immunoassay. In some examples, the immunoassay may comprise at least one antibody specific to human FMRP, for example, comprise two antibodies specific to the human FMRP, the two antibodies binding to different epitopes of the human FMRP.

Also within the scope of the present disclosure are a pharmaceutical composition for use in treating Fragile X syndrome (FXS) in a FXS patient who has undetectable peripheral Fragile X mental retardation protein (FMRP) or a level of peripheral FMRP no greater than (e.g., lower than) a predetermined value, wherein the pharmaceutical composition comprises an FXS agent (e.g., GABA_{A} modulator such as AZD7325, a muscle relaxer such as baclofen, and/or a vector for producing functional FMRP). Further, the present disclosure provides uses of an FXS agent (e.g., GABA_{A} modulator such as AZD7325, a muscle relaxer such as baclofen, and/or a vector for producing functional FMRP) for manufacturing a medicament for use in treating FXS in a FXS patient who has undetectable peripheral Fragile X mental retardation protein (FMRP) or a level of peripheral FMRP lower than a predetermined value.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a schematic illustration showing an exemplary experiment scheme for studying safety and efficacy of AZD7325 in FXS patients.

FIGS. 2A and 2B include diagrams showing top line results for RBANS list learning in FXS patients treated with low dose AZD7325, high dose AZD7325, or placebo. 2A: subgroup results for RBANS list learning. 2B: impact of low dose AZD7325 on RBANS in male patients with detectable or undetectable peripheral FMRP. For each of “Undetectable Male” and “Detectable Male,” the left bar refers to “Acute” and the right bar refers to “Chronic.”

FIG. 3 is a chart showing subtle variation in FMRP protein expression in FXS patients having no known dx, permutation, or full mutation. The FMRP concentrations are normalized to white blood cells.

FIG. 4 is a chart showing ABC irritability reduces with low dose AZD7325 regarding of FMRP status (trending).

FIG. 5 is a chart showing ABC lethargy/social withdrawal reduces in FMRP-absent males and increases in FMRP-present males at low dose AZD7325.

FIG. 6 is a chart showing FMRP-present males trending increase in WJ auditory attention.

FIGS. 7A-7I include charts showing low dose vs placebo in chronic trials in all subjects. 7A: chronic changes in theta band in patients with nondetectable FMRP. 7B: chronic changes in theta band in male patients with detectable FMRP. 7C: chronic change in theta band in female patients with detectable FMRP. 7D: chronic change in beta band in patients with nondetectable FMRP. 7E: chronic change in beta band in male patients with detectable FMRP. 7F: chronic change in beta band in female patients with detectable FMRP. 7G: chronic change in gamma 1 band in patients with nondetectable FMRP. 7H: Chronic change in gamma 1 band in male patients with detectable FMRP. 7I: Chronic change in gamma 1 band in female patients with detectable FMRP.

FIGS. 8A and 8B include diagrams summarizing electroencephalogram (EGG) signal changes in FXS patients with non-detectable peripheral FMRP. 10A: chronic trials. 10B: acute trials.

### DETAILED DESCRIPTION OF THE INVENTION

Fragile X Syndrome (FXS) also known as Martin-Bell syndrome or Escalante's syndrome, is a genetic disorder resulting from an expansion of the CGG trinucleotide repeat in the FMR1 gene on the X chromosome. The expanded CGG trinucleotide repeat responsible for FXS is located in the 5′ untranslated region (UTR) of the FMR1 gene, which encodes the fragile X mental retardation protein (FMRP), which is required for normal neural development. A trinucleotide repeat (CGG) in the 5′ UTR is normally found at 6-53 copies; however, individuals affected with FXS generally have 55-230 repeats of the CGG codon, which results in methylation of the FMR1 promoter, silencing of the gene, and a failure to produce FMR1 protein.

FMRP associates with hundreds of mRNAs regulating their translation and stability and can also directly affect neuronal excitability by binding ion channels at synapses. Consequently, loss of FMRP leads to a plethora of molecular, cellular and structural defects that are difficult, if not impossible, to correct with single-drug strategies in humans. The resulting defects occurring in the absence of FMRP can result in cognitive disability, communication deficits, social skill deficits, sensory sensitivity, inattention, adaptive behavior deficits, anxiety, autonomic system dysregulation, and seizure.

Reported herein, as an example, is a human clinical trial involving the use of AZD7325, a GABA_{A} modulator, for treating FXS patients. Unexpectedly, it was found that FXS patients having undetectable peripheral FMRP were responsive to a treatment involving AZD7325 at a low dose (e.g., 5 mg twice per day). Further, it was found that EEG values, such as gamma EEG values, were predictive of a subgroup of patients who were responsive to the low-dose AZD7325 treatment. Accordingly, peripheral FMRP, either taken alone or in combination with EEG values, can be used as a reliable biomarker for identifying FXS patients (e.g., male FXS patients) who are likely to be responsive to a low dose treatment comprising GABA_{A} stimulators such as AZD7325. In addition, EEG values, e.g., gamma EEG values, would be an indicia showing treatment efficacy of AZD7325 (e.g., at a low dose) in certain FXS patients.

Accordingly, provided herein are methods for identifying FXS patients for an FXS treatment using peripheral FMRP and/or gamma EEG as biomarkers and optionally subjecting the FXS patient to the FXS treatment. In some embodiments, the FXS treatment may comprise a GABA_{A} modulator such as AZD7325 (e.g., at a low dose).

I. Identification of FXS Patients for Treatment with GABA_{A} Modulators

In some aspects, provided herein is a method for identifying a Fragile X Syndrome (FXS) patient who is likely to respond to an FXS treatment, e.g., an FXS treatment comprising a GABA_{A} receptor modulator, using peripheral FMRP (non-brain FMRP) and/or EEG (e.g., gamma EEG) as a biomarker. Such a method may comprise detecting presence/absence of Fragile X mental retardation protein (FMRP) and/or measuring the level of FMRP in a biological sample of a candidate FXS patient. In some instances, undetectable FMRP in the biological sample indicates that the FXS patient is likely to respond to the FXS treatment, e.g., a treatment comprising a GABA_{A} receptor modulator. In other instances, a low level of FMRP in the biological sample, e.g., a level lower than a predetermined value, may be indicative of responsiveness of the FXS patient to the FXS treatment.

In some instances, EEG (e.g., gamma EEG) may be performed on the FXS patient to further determine responsiveness of the patient to the FXS treatment, e.g., a treatment comprising a GABA_{A} receptor modulator. The method may further comprise subjecting the FXS patient to the FXS treatment, for example, administering to the FXS patient thus identified an effective amount (e.g., a low dose) of the GABA_{A} receptor modulator. See detailed disclosures below.

The term “biomarker” as used herein refers to an indicator (one factor or a combination of factors) that provides information about clinical features of a FXS patient, for example, phenotypic severity of the disease, and/or patient responsiveness to a treatment. In certain embodiments, the biomarker can be a polypeptide, e.g., a FMRP polypeptide in a biological sample. In certain embodiments, the biomarker can be a parameter reflecting brain activity, for example, an electroencephalogram value such as gamma wave or alpha wave.

(a) Candidate FXS Patients

A candidate FXS patient may be a human patient (e.g., a male patient) diagnosed as having FXS by routine medical practice. For example, the candidate FXS patient may be an adult patient (e.g., ≥18). In some instances, the FXS patient may be between 18 to 50 years old. Such a candidate patient may be diagnosed as having full mutation of FXS, which can be determined using a conventional genetic testing. In some instances, the FXS patient may have an IQ less than or equal to 80. Alternatively or in addition, the FXS patient may have an Aberrant Behavior Checklist total score of 20 or higher.

In some embodiments, the candidate FXS patient is free of concomitant use of modulators of GABA A neurotransmission. The candidate patient may also be free of psychotropic drugs (e.g., more than three) that do not directly impact GABA transmission, and/or unstable dosing of any psychotropic medication prior to (e.g., 4 weeks prior to) performance of any of the identification methods disclosed herein. In some instances, the candidate patient is free of uses of strong and moderate modulators of CYP3A and CYP2C19 during the screening. Further, the candidate patients may be free of CNS-suppressing agents such as central analgesics, muscle relaxants, benzodiazepines, other sedatives, and/or alcohol. The candidate patient may also be free of unstable seizure disorder (see Example 1 below) and/or a change in any anti-convulsant drug dosing in the 60 days prior to performance of the methods disclosed herein.

Additional eligibility requirements may be found, for example, in Example 1 below.

(b) Detecting Peripheral FMRP Polypeptide

The presence/absence and/or level of a FMRP polypeptide may be detected in a biological sample of any of the candidate FXS patients disclosed herein by a conventional assay or an assay disclosed herein. The biological sample may be a biofluid sample. In some examples, the biological sample can be a blood sample, for example, a dried blood spot sample. In some examples, the biological sample can be a serum sample. In some examples, the biological sample can be a plasma sample.

The FMRP polypeptide to be detected or measured in the method disclosed herein may be a polypeptide expressed from a FMR1 gene (e.g., a mutated FMR1 gene). The FMR1 gene is a highly conserved gene that consists of 17 exons spanning approximately 38 kb of genomic DNA. The FMR1 gene undergoes extensive alternative splicing yielding different FMR1 transcriptional isoforms, resulting in several FMR1 protein isoforms. FMR1 transcriptional isoforms can be categorized into groups by their exon structures as shown in Table 1 below.

TABLE 1
Splice pattern grouping of FMR1 transcriptional isoforms

| Group | Exons |
|---|---|
| A | 9, 10, 11, 12, 13, 14, 15, 16, 17 |
| B | 9, 10, 11, 12, 13, 15, 16, 17 |
| C | 9, 10, 11, 13, 14, 15, 16, 17 |
| D | 9, 10, 11, 13, 15, 16, 17 |
| E | 9, 10, 15, 16, 17 |
| F | Different combination of exons |

The human FMR1 gene can produce a total of 11 FMR1 protein isoforms as a result of alternative splicing. These FMRP isoforms share a highly conserved N-terminal fragment of ˜400 residues and variable C-terminal sequences with varying mRNA-binding affinities. Any of the splice isoforms of FMR1 can be used in the present disclosure. In some examples, the human FRM1 protein used herein is FRM1 isoform 1. Exemplary coding sequence for the FMR1 protein can be found under GenBank accession no. NM_002024.

The FMRP polypeptide for detection/measurement may be any isoform produced by a FMR1 gene, for example, those listed in Table 1 above.

Presence/absence and/or levels of a FMRP polypeptide in any of the biological samples disclosed herein may be measured by conventional methods. In some instances, presence/absence and/or levels of a FMRP polypeptide may be measured by an immune assay, which refers to a biochemical assay for determining the presence or concentration of a target molecule through the use of an antibody or an antigen. Examples include, but are not limited to, enzyme-linked immunosorbent assays (ELISAs), Westernblot, radioimmunoassays (RIA), counting immunoassays (CIA), fluoroimmunoassays (FIA), and chemiluminescenceimmunoassays (CLIA). In some embodiments, an immune assay comprising an antibody specific to a FMRP polypeptide may be used.

In some examples, peripheral presence/absence and/or levels of a FMRP polypeptide in a biological sample such as a dried blood spot sample may be determined by an immune assay, which may involve two antibodies that bind different epitopes (e.g., non-overlapping epitopes) in the FMRP polypeptide. A Luminex® assay may be used in detecting the FMRP polypeptide in the biological sample. Additional information may be find in Example 3 below.

In some embodiments, a housekeeping marker may be co-measured. A housekeeping marker (e.g., a housekeeping gene or cell) are typically constitutive biological molecules or cells required for maintenance of basic cellular or organ function and are expressed or present in an organism under normal and patho-physiological conditions. In some examples, the housekeeping marker may be a type of blood cells, for example, white blood cells (WBCs). The row results relating to the presence/absence or level of a FMRP polypeptide may be normalized against the housekeeping marker to avoid experimental variations.

(c) Electroencephalogram (EEG)

An electroencephalogram (EEG) is a test that detects abnormalities in the brain waves, or in the electrical activity of the brain of a patient. During the procedure, electrodes comprising small metal discs with thin wires are pasted onto a patient's scalp. The electrodes detect tiny electrical charges that result from the activity of brain cells. The charges are amplified, recorded, and analyzed.

In some embodiments, gamma waves and/or alpha waves of a candidate FXS patient (or a FXS patient subject to the treatment disclosed herein) may be assessed by electroencephalogram (EEG) and can be used as a biomarker for assessing the patient's responsiveness to an FXS treatment (e.g., a treatment comprising a GABA_{A} modulator) and/or efficacy of the treatment.

In some embodiments, beta activity may be measured in an FXS patient before, during, and/or after the treatment disclosed herein by EEG. Beta activity may comprise EEG frequencies ranging from about 15 to about 30 Hz. Beta waves are believed to be associated with active, busy, or anxious thinking and active concentration. In some examples, beta activity of an FXS patient as disclosed herein may be monitored by EEG for assessing disease status, severity, and/or efficacy of a treatment.

In some embodiments, gamma activity may be measured in an FXS patient before, during, and/or after the treatment disclosed herein by EEG. Gamma activity may comprise EEG frequencies ranging from about 30 to about 90 Hz (e.g., about 30-60 Hz or about 60-90 Hz). Typically, it is distributed widely throughout cerebral structures. Gamma activity is associated with various cerebral functions, such as perception, attention, memory, consciousness, synaptic plasticity, and/or motor control. FXS patients often exhibit elevated gamma activity, which is indicative of noise, excessive synaptic connectivity, and/or brain cell over connecting). In some examples, gamma activity of an FXS patient as disclosed herein may be monitored by EEG for assessing disease status, severity, and/or efficacy of a treatment.

Alternatively or in addition, alpha activity and/or theta activity may be measured in an FXS patient before, during, and/or after the treatment disclosed herein by EEG. Alpha waves may comprise neutral oscillations in the frequency range of about 8-14 Hz. Theta waves often refer to frequency components in the 3-7 Hz range.

(d) Identification of FXS Patients for Treatment

A FXS patient suitable for a treatment comprising an FXS treatment, for example, those disclosed herein such as a treatment comprising a GABA_{A} modulator (e.g., AZD7325), can be identified based on the presence/absence or level of peripheral FMRP and/or EEG values (e.g., gamma EEG values) as disclosed herein. In some instances, the FXS treatment may comprise a GABA_{A} modulator, for example, a selective GABA_{A} stimulator. In some examples, the FXS treatment may comprise a low dose of any of the GABA_{A} modulator disclosed herein. See, e.g., disclosures below.

In some instances, a FXS patient (e.g., a male FXS patient such as a male adult FXS patient) who shows undetectable peripheral FMRP may be identified as suitable for (i.e., likely to respond to) the treatment disclosed herein. “Undetectable peripheral FMRP” means that the presence of a FMRP polypeptide cannot be detected in a conventional assay (e.g., an immune assay as disclosed herein) as determined by a skilled person in the art. For example, if no signal above a background signal is revealed in the assay, it can be determined that the FMRP polypeptide is undetectable.

In some instances, a FXS patient (e.g., a male FXS patient such as a male adult FXS patient) who shows a low level of peripheral FMRP may be identified as suitable for (i.e., likely to respond to) the treatment disclosed herein. A low level of peripheral FMRP means that the level of peripheral FMRP in a candidate FXS patient is substantially lower than others such that it would have expected that brain distribution of the FMRP polypeptide is essentially free (e.g., no brain distribution or very low level of brain distribution such that meaningful bioactivity of FMRP in the brain would be expected). In some examples, a low level of peripheral FMRP may be determined by comparing the level of peripheral FMRP in a candidate FXS patient with a predetermined value. If the level of peripheral FMRP of the candidate FXS patient is no greater than or lower than the predetermined value, it indicates that the candidate FXS patient is likely to respond to the FXS treatment as disclosed herein. The predetermined value may be a cutoff value representing the level of peripheral FMRP in FXS patients who have essential free presence of FMRP in the brain. Such a cutoff value can be determined via conventional approaches by medical practitioners. See, e.g., Example 1 below.

In some embodiments, the peripheral FMRP level of an FXS patient may be determined using dried blood spot samples and an immune assay such as a Luminex® Assay. See Examples 1 and 3 below. The FMRP concentrations thus determined are estimated to be about 7 folds higher than the blood level of FMRP in that patient. In some instances, a peripheral FMRP level lower than about 5 pM as determined by the method provided herein may be identified as likely to respond to the FXS treatment disclosed herein (e.g., comprising a GABA_{A} modulator, for example, a selective GABA_{A} stimulator (e.g., AZD7325) or comprising a muscle relaxer such as baclofen). For example, an FXS patient having a peripheral FMRP level lower than about 3 pM may be identified as likely to respond to the FXS treatment disclosed herein. In another example, an FXS patient having a peripheral FMRP level lower than about 2 pM may be identified as likely to respond to the FXS treatment disclosed herein. In yet another example, an FXS patient having a peripheral FMRP level lower than about 1.5 pM may be identified as likely to respond to the FXS treatment disclosed herein. Alternatively, an FXS patient having a peripheral FMRP level lower than about 1 pM may be identified as likely to respond to the FXS treatment disclosed herein. In another example, an FXS patient having a peripheral FMRP level lower than about 0.5 pM may be identified as likely to respond to the FXS treatment disclosed herein.

In some examples, the results of a FMRP polypeptide may be normalized against a housekeeping marker and whether the FMRP polypeptide is detectable or undetectable can be determined based on the normalized result.

### II. Treatment of FXS Patients

Also provided herein is a method for treating a FXS patient who has low or undetectable peripheral FMRP with an FXS agent, such as a GABA_{A} modulator (e.g., a selective GABA_{A} stimulator such as AZD7325). In some embodiments, the FXS patient has undetectable peripheral FMRP. The FXS patient to be treated may be identified by a method as disclosed herein, e.g., using peripheral FMRP, optionally in combination with EEG values such as gamma EEG values as a marker.

(a) GABA_{A} Modulators

The GABA_{A} receptors are a class of receptors that are responsive to the neurotransmitter gamma-aminobutyric acid (GABA), which is an inhibitory molecule for the vertebrate central nervous system. GABA_{A} receptors are ligand-gated ion channels.

In some embodiments, the GABA_{A} modulator for use in the methods disclosed herein may be a positive allosteric modulator (PAM), which increases the activity of a GABA_{A} receptor. GABA is a major inhibitory neurotransmitter in the central nervous system. Upon binding, it triggers the GABA_{A} receptor to open its chloride channel to allow chloride ions into the neuron, making the cell hyperpolarized and less likely to fire. GABA_{A} PAMs increase the effect of GABA by making the channel open more frequently or for longer periods. Examples of GABA_{A} PAMs include, but are not limited to, alcohols (e.g., ethanol, or isopropanol); avermectins (e.g., ivermectin); barbiturates (e.g., phenobarbital); benzodiazepines (e.g., diazepam, or alprazolam); bromides (e.g., potassium bromide); carbamates (e.g., meprobamate, or carisoprodol); chloral hydrate, chloralose, petrichloral, and other 2,2,2-trichloroethanol prodrugs; chlormezanone; clomethiazole; dihydroergolines (e.g., ergoloid such as dihydroergotoxine); disulfonylalkanes (e.g., sulfonmethane, tetronal, or trional); etazepine; etifoxine; 2-Substituted phenols (e.g., thymol, or eugenol); imidazoles (e.g., etomidate); kavalactones; loreclezole; neuroactive steroids (e.g., allopregnanolone, ganaxolone); nonbenzodiazepines (e.g., zaleplon, zolpidem, zopiclone, or eszopiclone); propofol; piperidinediones (e.g., glutethimide, or methyprylon); propanidid; pyrazolopyridines (e.g., etazolate); quinazolinones (e.g., methaqualone); skullcap constituents; stiripentol; valerian constituents (e.g., valeric acid, or valerenic acid); or volatile organic compounds (e.g., chloroform, diethyl ether, or sevoflurane).

In some examples, the GABA_{A} modulator is a selective PAM, for example, AZD7325. AZD7325 is a partial selective PAM of GABAAα2 and Aα3 receptors and has less antagonistic activity against the Aα1 and Aα5 subtypes. The chemical structure of AZD7235 (4-amino-8-(2-fluoro-6-methoxyphenyl)-N-propylcinnoline-3-carboxamide) is shown below:

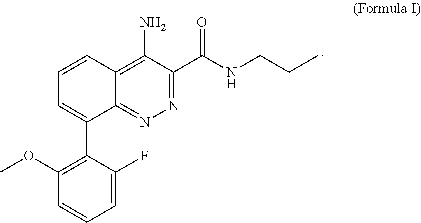 [SMILES: CCCNC(=O)c1nnc2c(-c3c(F)cccc3OC)cccc2c1N]

Prodrugs, derivatives, and metabolites of AZD7325 are also within the scope of the present disclosures.

In other embodiments, the GABA_{A} modulator is a GABA_{A} agonist, which activates a GABA_{A} receptor. A GABA_{A} agonist typically would produce sedative effects and may also cause other effects such as anxiolytic, anticonvulsant, and muscle relaxant effects. Examples include, but are not limited to, abecarnil, barbiturates (e.g., in high doses), bamaluzole, eszopiclone, fengabine, GABA, gabamide, GABOB, gaboxadol, ibotenic acid, isoguvacine, isonipecotic acid, muscimol, pantherine, phenibut, picamilon, progabide, propofol, quisqualamine, SL 75102, thiomuscimol, topiramate, or zolpidem.

In another example, the FXS therapy may comprise a muscle relaxer, for example, baclofen (e.g., racemic baclofen).

Other FXS therapies, such as those known in the art or disclosed herein, are also within the scope of the present disclosure. Non-limiting examples include gene therapy (e.g., delivering a gene encoding a functional FMRP polypeptide via a proper vehicle, e.g., a viral vector such as an AAV vector), antidepressants (e.g., selective serotonin reuptake inhibitors (SSRIs), venlafaxine, or tricyclic antidepressants), atypical antipsychotics such as alpha 2-agonists (e.g., clonidine and guaneficine), risperidone, quetiapine, ziprasidone, valproic acid, and carbamazepine. Any of the FXS medications disclosed herein may be used in combination with educational, behavioral, or physical therapy.

(b) Pharmaceutical Compositions

Any of the FXS agent such as the GABA_{A} modulators (e.g., AZD7325) disclosed herein may be formulated to form a pharmaceutical composition, which may further comprise a pharmaceutically acceptable carrier, diluent or excipient. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20 Ed. (2000) Lippincott Williams and Wilkins, Ed. K. E. Hoover.

In some embodiments, the pharmaceutical compositions or formulations are for parenteral administration, such as intravenous, or oral administration. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating any of the GABA_{A} modulators (e.g., AZD7325) in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The pharmaceutical compositions disclosed herein may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween®, pluronics or polyethylene glycols.

(c) Treatment of Suitable FXS Patients

Any of the FXS agents, such as GABA_{A} modulators (e.g., AZD7325) or a pharmaceutical composition comprising such as disclosed herein can be used to treat a suitable FXS patient as also disclosed herein, e.g., an adult human FXS (e.g., male FXS patient) having low or undetectable peripheral FMRP polypeptides. In some embodiments, the suitable FXS patient has undetectable peripheral FMRP. Additional features of an FXS suitable for the treatment disclosed herein are provided elsewhere herein, for example, Section I(a) above and/or Example 1 below.

To perform the method disclosed herein, an effective amount of the FXS agent such as GABA_{A} modulator (e.g., AZD7325) or a pharmaceutical composition comprising such may be administered to a subject who needs treatment via a suitable route (e.g., intravenous, or oral administration) at a suitable amount as disclosed herein.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

A subject to be treated by any of the methods disclosed herein may be a human patient having FXS, who can be identified by routine medical examination, e.g., laboratory tests, organ functional tests, behavioral tests, CT scans, electroencephalogram, magnetic resonance imaging (MRI), and/or genetic test. FXS patients typically have one or more genetic mutations in the FMR1 gene, which usually makes a protein called fragile X mental retardation protein (FMRP), also referred to as FMR1 protein. Nearly all cases of fragile X syndrome are caused by a mutation, in which a DNA segment, known as the CGG triplet repeat, is expanded within the FMR1 gene. Normally, this DNA segment is repeated from 5 to about 40 times. In patients with FXS, the CGG segment is repeated more than 200 times. The abnormally expanded CGG segment turns off (silences) the FMR1 gene, which prevents the gene from producing FMRP. Males and females with 55 to 200 repeats of the CGG segment are said to have an FMR1 gene premutation. Most people with this premutation are intellectually normal. In some cases, however, individuals with a premutation have lower than normal amounts of FMRP. As a result, they may have mild versions of the physical features seen in FXS. FXS is inherited in an X-linked dominant pattern. The inheritance is dominant if one copy of the altered gene in each cell is sufficient to cause the condition. X-linked dominant means that in females (who have two X chromosomes), a mutation in one of the two copies of a gene in each cell is sufficient to cause the disorder. In males (who have only one X chromosome), a mutation in the only copy of a gene in each cell causes the disorder. In most cases, males experience more severe symptoms of the disorder than females.

An FXS patient who are likely to respond to an FXS treatment as disclosed herein, for example, a treatment comprising a GABA_{A} modulator such as AZD7325, can be identified by any of the identification methods disclosed herein. In some embodiments, the patient may be a human adult FXS patient. In some embodiments, the subject may be a male human adult FXS patient. Such an adult patient may be ≥18, and optionally ≤50.

Alternatively or in addition, the FXS patient to be treated in the methods disclosed herein may carry an expanded CGG segment within the FMR1 gene. In some examples, a FXS patient may carry an expanded CGG segment repeated more than 200 times within the FMR1 gene. In some examples, a FXS patient may be a male patient having an X-linked mutation in the FMR1 gene. In some embodiments, patients suspected of having or at risk of having FXS with at least one FMR1 gene permutation may be treated with the methods disclosed herein. Genetic testing can be performed to a candidate subject using routine generation sequencing methods, including, but not limited to, next-generation sequencing, pyrosequencing, Sanger sequencing, whole exome sequencing, whole genome sequencing, and the like.

In any of the methods disclosed herein, an effective amount of the GABA_{A} receptor modulator such as AZD7325 can be given to a suitable FXS patient as disclosed herein to alleviate one or more symptoms associated with FXS. In some instances, symptoms associated with FXS may be behavioral, cognitive neurorehabilitation, or a combination thereof. In some examples, symptoms of FXS can be anxiety-related and perseverative behaviors, social behaviors, learning, memory, or a combination thereof.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Effective amounts can also vary, depending on phenotypic variability among subjects having FXS, and/or the genetic mutations involved.

In some embodiments, a treatment method as disclosed herein may comprise a low dose of the GABA_{A} receptor modulator, which refers to a dose lower than the commonly used dose for that particular GABA_{A} receptor modulator (e.g., at least 50%, at least 80%, at least 2-fold, at least 5-fold, or at least 10-fold lower). For example, the treatment method disclosed herein may comprise oral administration of a low dose of AZD7325, which may range from about 5-20 mg per day. In some examples, the daily dose of AZD7325 may range from about 6-15 mg. In some examples, the daily dose of AZD7325 may be about 8-10 mg. The daily dose may be given to a suitable FXS patient once per day, twice per day, or three times per day. In some examples, AZD7325 is given to a suitable FXS patient orally at about 1 to about 7.5 mg twice per day. In one specific example, AZD7325 is given to a suitable FXS patient orally at about 5 mg twice per day.

Also within the present disclosure is a method for treating an FXS patient comprising administering to the FXS patient a low dose of a GABA_{A} receptor modulator such as AZD7325. In some embodiments, the method may comprise administering to an FXS patient AZD7325 at a daily dose of about 5-20 mg (e.g., about 6-15 mg, or about 8-10 mg). In some examples, the method may comprise administering to an FXS patient AZD7325 at 5 mg twice per day, e.g., orally.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value. In some embodiments, the hinge domain is a hinge domain of a naturally occurring protein.

Conventional methods, known to those of ordinary skill in the art of medicine, can be used to administer any of the FXS agent such as a GABA_{A} receptor modulator (e.g., AZD7325) or a pharmaceutical composition comprising such to the FXS patient. For example, this pharmaceutical composition can also be administered parenterally, e.g., by intravenous injection or by oral administration.

In some embodiments, the FXS patient to be treated by the method described herein may be a human patient who has undergone or is subjecting to another FXS therapy. The other FXS therapy may be complete. Alternatively, the other FXS therapy may be still ongoing. In other embodiments, the FXS patient may be subject to a combined therapy involving the GABA_{A} receptor modulator (e.g., AXD7325) disclosed herein and a second FXS therapy (e.g., a gene therapy involving delivery of a FMR1 gene via, e.g., an AAV vector). FXS treatments include, but are not limited to, treatment of behavioral abnormalities, seizures, speech therapy, physical therapy, and so forth. Exemplary FXS treatments include, but are not limited to, treatment comprising a PI3K isoform-selective inhibitor, a MMP9 antagonist, or a combination thereof. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

In some embodiments, the FXS patient may be subject to EEG (e.g., gamma EEG) before, during the course, or after the treatment comprising any of the GABA_{A} receptor modulators disclosed herein (e.g., AZD7325). Change of the EEG values (e.g., gamma waves) may be used for assessing treatment efficacy. For example, a reduction of gamma EEG values is indicative of effectiveness of the treatment.

III. Kits for Identifying and/or Treating FXS Patients

The present disclosure also provides kits for use in identifying a suitable FXS patient for the FXS treatment such as a treatment comprising a GABA_{A} receptor modulator (e.g., AZD7325) and/or for treating FXS as described herein.

In some embodiments, provided herein is a kit for determining whether a candidate FXS patient has detectable or undectable peripheral FMRP, which can be relied on to determine whether the FXS patient is likely to respond to the FXS treatment (e.g., comprising a GABA_{A} receptor modulator treatment, such as a treatment comprising AZD7325). Such a kit may comprise one or more containers in which one or more agents for detecting a FMRP polypeptide may be placed.

In other embodiments, provided herein is a kit for treating a suitable FXS patient, who may be identified by any of the identification methods disclosed herein. Such a kit may comprise one or more contains in which an FXS therapeutic agent (e.g., GABA_{A} modulator such as AZD7325) may be placed.

Any of the kits disclosed herein may additionally comprise instructions for use of therapeutic agent in any of the methods described herein. The included instructions may comprise a description of administration of the FXS agent such as a GABA_{A} receptor modulator (e.g., AZD7325) or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the GABA_{A} receptor modulator (e.g., AZD7325) or the pharmaceutical composition comprising such to a subject who has or is suspected of having FXS.

The instructions relating to the use of the GABA_{A} receptor modulator (e.g., AZD7325), the muscle relaxer such as baclofen, and/or the gene therapy agents (e.g., a vector such as an AAV vector for producing a functional FMRP polypeptide) as described herein generally include information as to dosage, dosing schedule, and route of administration for the intended treatment. In some embodiments, the instructions comprise a description of optimizing the dose of the active agent (e.g., those disclosed herein) in a subject having FXS using one or more of the behavior features as a biomarker, e.g., those described herein. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Alternatively, the kit may additionally comprise instructions for use of the detecting agent. For example, the included instructions may comprise a description of how to use the detecting agent for measuring presence/absence or level of a FMRP polypeptide in a biological sample.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Study of AZD7325 in Adults with Fragile X Syndrome

This study aims at investigating the safety, tolerability, and blood pharmacodynamics of treatment with oral administration of AZD7325 in adults with Fragile X Syndrome (FXS). This study also investigates measures of efficacy and biomarkers during treatment.

### Study Design

The treatment conditions are provided Table 2 below:

TABLE 2
Study Design

| Condition or Disease | Intervention/Treatment |
|---|---|
| Fragile X | AZD7325 5 mg (low dose), twice per day; |
| Syndrome (FXS) | oral in gelatin capsules |
| | AZD7325 15 mg (high dose), twice per day; |
| | oral in gelatin capsules |
| | Placebo oral capsule (dosed similar to AZD7235 |
| | in terms of dosage form, frequency and duration) |

Primary Outcome Measures:
Amyloid precursor protein (APP) through the end of the study, approximately 12 weeks;
Short-term treatment of peripheral APP dysregulation by correcting elevated levels.

Secondary Outcome Measures:
Change in the social withdrawal subscale score of the Aberrant Behavior Checklist (ABC) through the end of the study, approximately 12 weeks. The ABC is the gold standard parent/caregiver reported behavioral outcome measure for use in development disability clinical trials.
Change in the Pediatric Anxiety Rating Scale (PARS) through the end of the study, approximately 12 weeks. The PARS is the gold standard parent/caregiver reported anxiety outcome measure for use in Fragile X Syndrome clinical trials.

Inclusion Criteria:
Diagnostic confirmation of full mutation FXS
18 to 50 years old, males and females
General good health as determined by physical example, medical history and laboratory work up
FXS genetic report at screening
IQ less than or equal to 80. IQ cutoff is used as a means to exclude cases of females with FXS who have the full mutation, but may have neurotypical development (i.e., do not have the full FXS phenotype despite positive FXS genetic testing) due to variability in X chromosome inactivation patterns.
Male study participants who are sexually active with a female partner of childbearing potential must be surgically sterilized, practicing abstinence, or agree to use highly effective methods of birth control (defined in the list below), and not rely on barrier methods and spermicide alone, from the time of screening until 1 week after final dose of study drug. Male study participants must also not donate sperm from the time of screening until 1 week after final dose of study drug. Given that AZD7325 is not mutagenic, there is no mandatory requirement for condom use, either for avoidance of procreation or in the case of treated males with a pregnant partner.
Women of childbearing potential may be included in the study provided they are established on, and continue to use, highly effective contraceptive methods from the time of screening until 1 week after the final dose of study drug. Highly effective methods of contraception associated with inhibition of ovulation (either oral, intravaginal or transdermal), progestin-only hormonal contraception associated with inhibition of ovulation (either oral [specifically Micronor, Nor-QD or their generic equivalents], injectable or implantable).
Aberrant Behavior Checklist total score of 20 or higher at screening.

Exclusion Criteria:
Concomitant use of modulators of GABA A neurotransmission. (examples)
Use of more than three psychotropic drugs that do not directly impact GABA transmission, and/or unstable dosing of any psychotropic medication in the 4 weeks prior to baseline visit.
Subjects are prohibited from use of strong and moderate modulators of CYP3A and CYP2C19 during the screening (at least 2 weeks before initiation of the study) and treatment periods of the study. Such prohibited drugs are outlined in fda.gov/downloads/drugs/guidancecomplianceregulatoryinfonnation/guidances/ucm292362.pdf
CNS-suppressing agents such as central analgesics, muscle relaxants, benzodiazepines, other sedatives, and should also limit alcohol intake to ≤1 alcoholic beverage per day.
Unstable seizure disorder as defined by any seizure in the 6 months prior to baseline visit and/or a change in any anti-convulsant drug dosing in the 60 days prior to study entry.
All patients with abnormal baseline safety lab assessments including, but not limited to ALT or AST greater than 1.5 the upper limit of normal, total bilirubin or creatinine greater than 1 time the upper limit of normal or other clinically relevant lab abnormality or abnormality in ECG, HR or BP at screening as judged by the investigator.
Clinical relevant history or presence of any medical disorder judged by the investigator at potentially interfering with this trial.
History of or current abuse of drugs or alcohol including prescription medication.
For female subjects of child bearing potential (women 50 & under is “amenorrhoeic for 12 months or more (following cessation of exogenous hormonal treatments—if these have been previously taken) and with luteinizing hormone (LH) and follicle stimulating hormone (FSH) levels in the post-menopausal range) a positive pregnancy test.

### Results

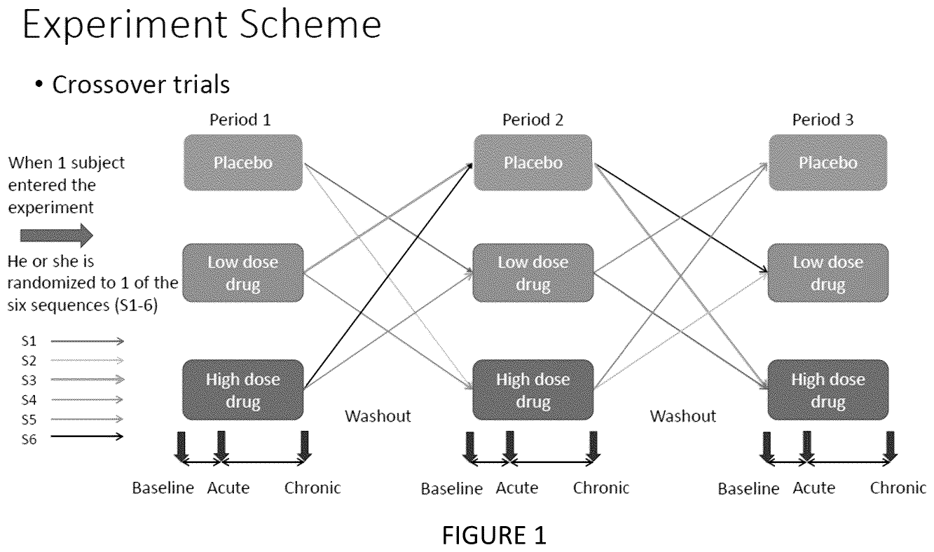

Fifteen subjects (11 males and 4 females) were initially scheduled as participants in this study. Among them, three subjects did not complete the entire experiment. An exemplary experimental scheme is provided in FIG. 1.

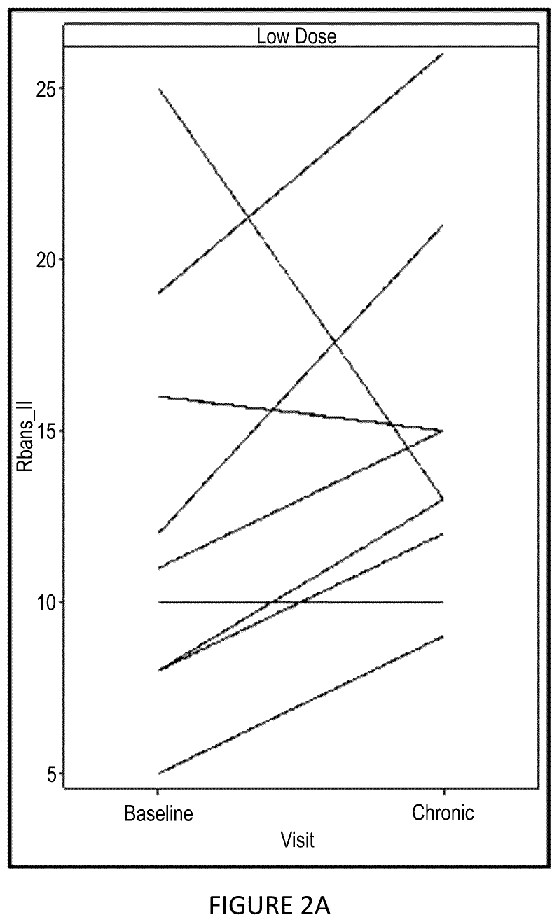

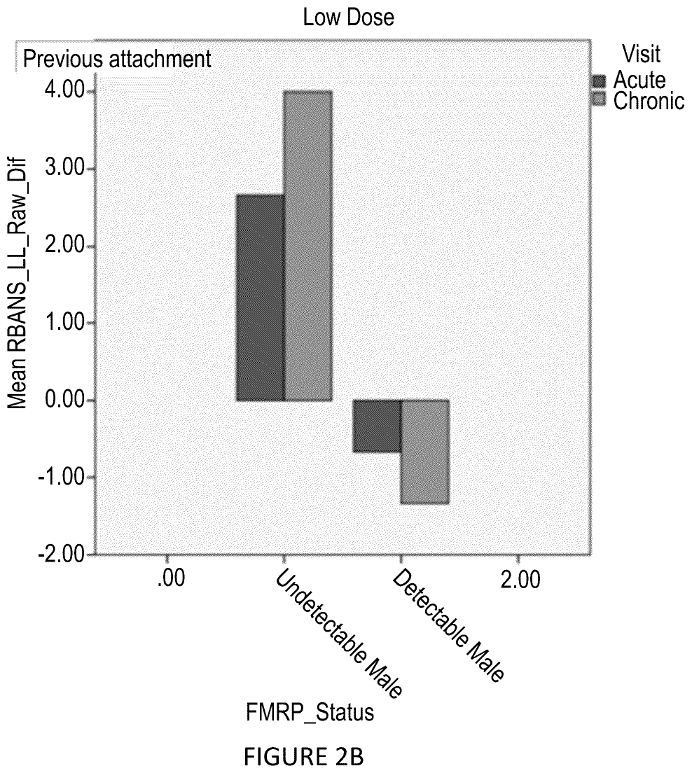

Repeatable Battery for the Assessment of Neuropsychological Status (RBANS) Results Tope line results for Repeatable Battery for the Assessment of Neuropsychological Status (RBANS) list learning includes: (1) a trending effect of drug was identified across all eligible subjects (p=0.09); (2) The trend indicates that low dose might improve RBANS list learning scores; (3) A significant beneficial effect of low-dose drug was identified in a subgroup of males following removal of an outlier; and (4) High dose displays dose limiting effect on performance on RBANS. Subgroup results for RBANS list learning are provided in Table 3 below. See also FIG. 2A. Low dose AZD7325 was observed as associated with improved memory in males as measured by the RBANS assay. See FIG. 2B.

TABLE 3
Subgroup Results for RBANS List Learning
Difference of Drug* Type least Squares Means

| Drug | Estimate | SEM | DF | t Value | Pr > [t] | Interpret |
|---|---|---|---|---|---|---|
| Baseline - | 1.24 | 1.42 | 36.9 | 0.88 | 0.38 | a |
| Placebo | | | | | | |
| Baseline -Low | −2.75 | 1.22 | 36.1 | −2.25 | 0.03 | b |
| Baseline-High | 2.09 | 1.41 | 36.9 | 1.48 | 0.15 | c |
| Placebo-Low | −4.00 | 1.84 | 39.3 | −2.17 | 0.04 | d |
| Placebo-High | 0.84 | 1.98 | 39.3 | 0.43 | 0.67 | e |
| Low-High | 4.84 | 1.84 | 39.3 | 2.63 | 0.01 | f |

a No change from baseline with placebo in score
b Low dose in this subgroup demonstrated an increase (benefit) of +2.75 of RBANS LL from baseline
c No change from baseline with high dose (direction is toward worsening, −2)
d Low dose demonstrated increase of nearly 4 (benefit) compared to placebo
e Low dose had greater score increase (benefit) than high dose, separating groups.

Protein Expression

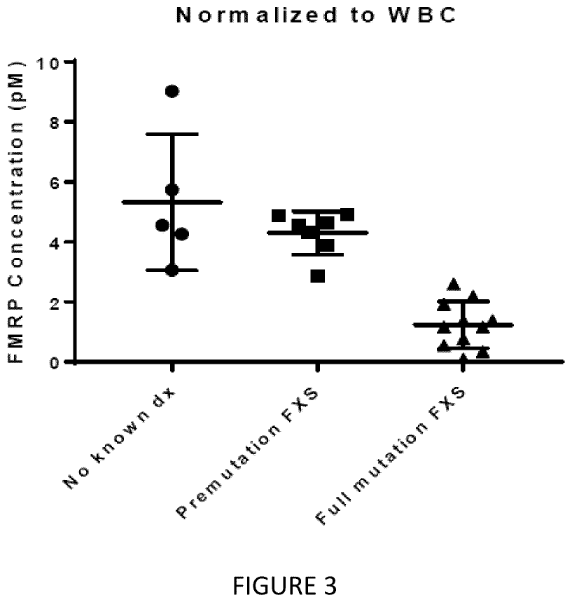

Peripheral levels FMRP proteins in the patients were measured by an immunoassay using an antibody specific to FMRP. A fair percentage of males with FXS show mosaicism or a mix of permutation and mutation cells. FIG. 3. Peripheral blood FMRP levels from the study participants are shown in Table 4 below (measured using dried blood spot samples using Luminex® Assay; see Example 3 below). The levels of FMRP measured using dried blood spot as disclosed herein are estimated to be about 7 folds higher than the FMRP levels in the blood of the same patient.

Five patients showed undetectable peripheral FMRP (FMRP value=0). Patients having a FMRP value below about 5 pM (e.g., below about 3 pM, below about 2 pM, below about 1 pM, or lower) as determined by the assay used in this example can be deemed as having a low level of peripheral FMRP.

TABLE 4
Peripheral Blood FMRP Levels in Study Participants

| Patient ID | FMRP Value (pM) |
|---|---|
| 008 | 15.47 |
| 012 | 0 |
| 010 | 2.88 |
| 001 | 1.98 |
| 002 | 0 |
| 003 | 1.89 |
| 005 | 0 |
| 007 | 13.96 |
| 009 | 27.26 |
| 004 | 0 |
| 006 | 0.57 |
| 015 | 8.43 |
| 014 | 0 |

Treatment Efficacy

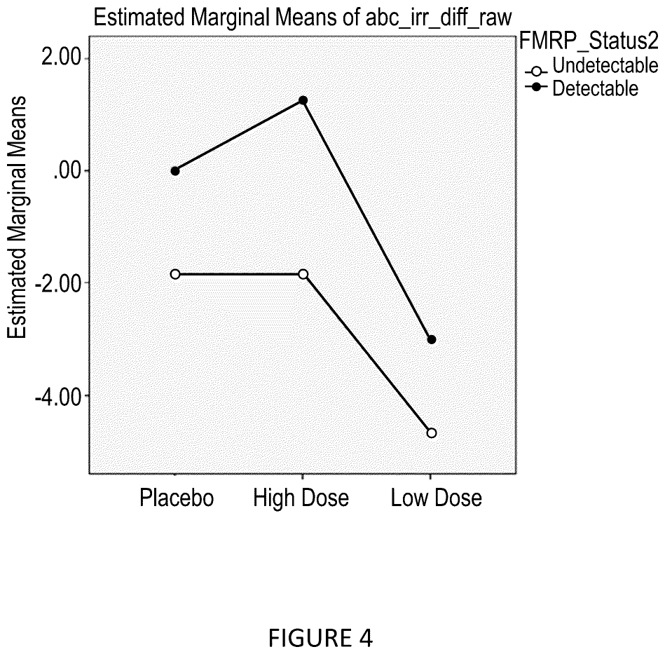

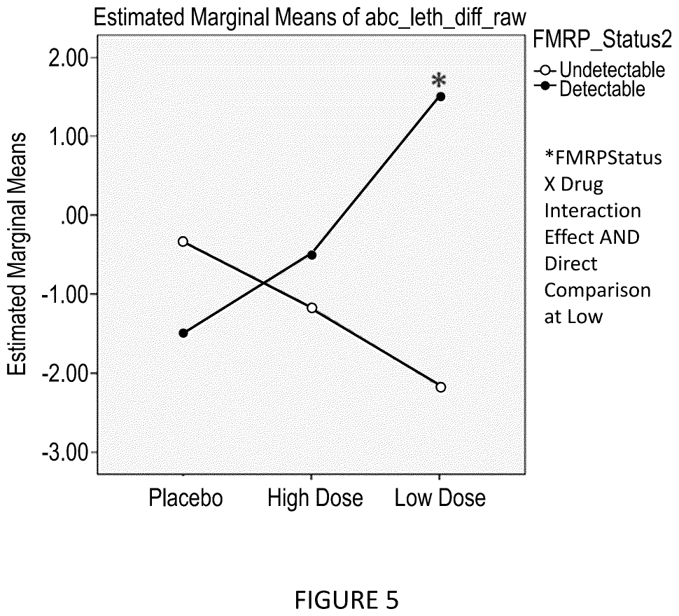

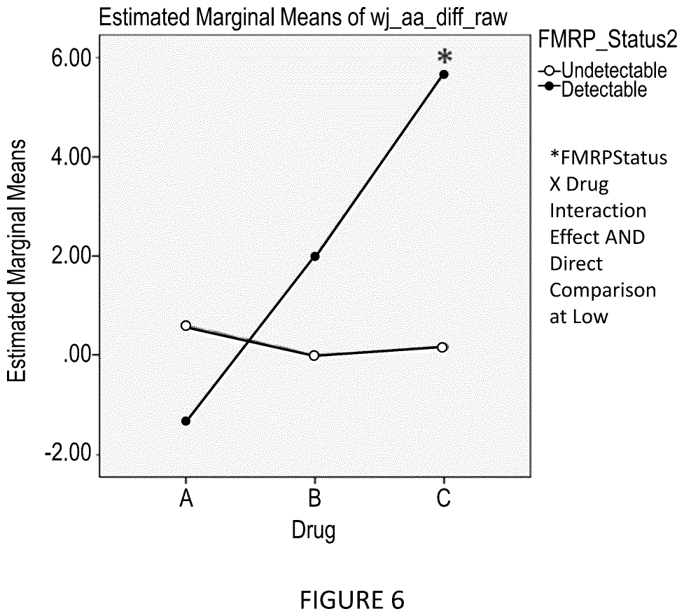

Low dose AZD7325 treatment resulted in Aberrant Behavior Checklist (ABC) irritability reduction, regardless of FMRP status (trending). See FIG. 4, comparing raw difference change from baseline to chronic. Further, low dose AZD7325 treatment led to reduction in ABC lethargy and social withdrawal in patients having undetectable peripheral FMRP. By contrast, low dose AZD7325 treatment led to increase in ABC lethargy and social withdrawal in patients having detectable peripheral FMRP. FIG. 5. FRMP-present male patients showed increase in WJ auditory attention as shown in FIG. 6.

Further RBANS List Learning results and ABC results from the 5 male FXS patients having undetectable peripheral FMRP are provided below.

(a) RBANS List Learning Sores

Table 5 below summarizes clinical measure RBANS List Learning scores from the 5 “undetectable” FMRP male subjects. RBANS List Learning is a verbal working memory task and raw scores provided indicate number of words remembered, with higher scores indicating more number of words remembered. Group average and standard deviation were obtained cross subjects per treatment type (Placebo/Low dose/High dose) and per visit, which are 1) baseline: before intake, 2) acute: after the first dosage, and 3) chronic: post 2-week daily dosage. Higher scores are clinically better.

TABLE 5
RBANS List Learning Scores in
Undetectable FMRP Male Subjects

| | Placebo | Low Dose | High Dose |
|---|---|---|---|
| RBANS-LL | (mean ± SD) | (mean ± SD) | (mean ± SD) |
| Baseline | 12.6 ± 5.73 | 10.6 ± 5.22 | 14.8 ± 8.32 |
| Acute | 12.8 ± 3.96 | 13.2 ± 3.11 | 10.4 ± 7.40 |
| Chronic | 13.4 ± 6.35 | 14.6 ± 6.80 | 12.2 ± 6.83 |

(b) Aberrant Behavior Checklist (ABC)

The Aberrant Behavior Checklist (ABC) is a standardized rating scale used for assessing problematic behavior designed for use in developmental disabilities, with validity for use in FXS (CITE). ABC-C is the original factor structure with 5 subscales: Irritability, Lethargy, Stereotypy, Hyperactivity, and Inappropriate Speech. See, e.g., Aman et al., J. Child Adolesc Psychopharmacol. 30(8):512-521 (2020), the relevant disclosures of which are incorporated by reference for the subject matter and purpose referenced herein. ABC was only assessed chronically (two weeks after treatment). Overall, no indication that ABC changed during chronic phase. The results are shown in Tables 6-10.

TABLE 6
Subscale 1 -- Irritability

| | Placebo | Low Dose | High Dose |
|---|---|---|---|
| ABC_C_Subscale_1 | (mean ± SD) | (mean ± SD) | (mean ± SD) |
| Baseline | 4.4 ± 3.36 | 7.6 ± 7.44 | 7.6 ± 7.44 |
| Chronic | 3.2 ± 5.07 | 7.4 ± 8.02 | 7.2 ± 5.36 |

TABLE 7
Subscale 2 - Lethargy

| | Placebo | Low Dose | High Dose |
|---|---|---|---|
| ABC_C_Subscale_2 | (mean ± SD) | (mean ± SD) | (mean ± SD) |
| Baseline | 2.4 ± 2.88 | 3.6 ± 2.97 | 3.2 ± 3.11 |
| Chronic | 1.0 ± 1.00 | 4.6 ± 2.70 | 1.8 ± 1.48 |

TABLE 8
Subscale 3 - Stereotypy

| | Placebo | Low Dose | High Dose |
|---|---|---|---|
| ABC_C_Subscale_3 | (mean ± SD) | (mean ± SD) | (mean ± SD) |
| Baseline | 4.4 ± 3.29 | 3.8 ± 2.28 | 5.0 ± 1.87 |
| Chronic | 3.0 ± 2.74 | 4.6 ± 3.05 | 3.4 ± 0.55 |

TABLE 9
Subscale 4 - Hyperactivity

| | Placebo | Low Dose | High Dose |
|---|---|---|---|
| ABC_C_Subscale_4 | (mean ± SD) | (mean ± SD) | (mean ± SD) |
| Baseline | 7.6 ± 10.33 | 7.8 ± 7.16 | 9.4 ± 10.74 |
| Chronic | 5.6 ± 6.95 | 7.2 ± 8.07 | 7.0 ± 9.06 |

TABLE 10
Subscale 5 - Inappropriate Speech

| | Placebo | Low Dose | High Dose |
|---|---|---|---|
| ABC_C_Subscale_5 | (mean ± SD) | (mean ± SD) | (mean ± SD) |
| Baseline | 5.6 ± 5.13 | 5.4 ± 4.16 | 5.6 ± 4.56 |
| Chronic | 4.0 ± 3.54 | 4.6 ± 3.29 | 4.4 ± 3.78 |

Tables 11-16 below summarize alternative EEG scoring for the FXS patient treated with AZD7325. ABC-FXS was created for use within this population to take into account FXS-specific symptoms, and has six subscales: Irritability, Social Unresponsiveness/Lethargy, Stereotypy, Hyperactivity, Inappropriate Speech, and Social Avoidance.

TABLE 11
Subscale 1 - Irritability

| | Placebo | Low Dose | High Dose |
|---|---|---|---|
| ABC_FXS_Subscale_1 | (mean ± SD) | (mean ± SD) | (mean ± SD) |
| Baseline | 6.8 ± 5.97 | 9.8 ± 8.23 | 10.2 ± 10.52 |
| Chronic | 4.4 ± 6.43 | 9.4 ± 9.99 | 9.2 ± 7.05 |

TABLE 12
Subscale 2 - Socially Unresponsive/Lethargic

| | Placebo | Low Dose | High Dose |
|---|---|---|---|
| ABC_FXS_Subscale_2 | (mean ± SD) | (mean ± SD) | (mean ± SD) |
| Baseline | 1.4 ± 2.07 | 2.4 ± 1.52 | 1.8 ± 1.92 |
| Chronic | 1.2 ± 0.84 | 2.2 ± 2.39 | 1.0 ± 1.22 |

TABLE 13
Subscale 3 - Stereotypy

| | Placebo | Low Dose | High Dose |
|---|---|---|---|
| ABC_FXS_Subscale_3 | (mean ± SD) | (mean ± SD) | (mean ± SD) |
| Baseline | 4.4 ± 3.29 | 3.8 ± 2.28 | 5.0 ± 1.87 |
| Chronic | 3.0 ± 2.74 | 4.6 ± 3.05 | 3.4 ± 0.55 |

TABLE 14
Subscale 4 - Hyperactivity

| | Placebo | Low Dose | High Dose |
|---|---|---|---|
| ABC_FXS_Subscale_4 | (mean ± SD) | (mean ± SD) | (mean ± SD) |
| Baseline | 4.8 ± 6.61 | 4.2 ± 3.77 | 6.4 ± 6.80 |
| Chronic | 3.8 ± 4.49 | 4.4 ± 4.98 | 4.6 ± 6.31 |

TABLE 15
Subscale 5 - Inappropriate Speech

| | Placebo | Low Dose | High Dose |
|---|---|---|---|
| ABC_FXS_Subscale_5 | (mean ± SD) | (mean ± SD) | (mean ± SD) |
| Baseline | 5.6 ± 5.13 | 5.4 ± 4.16 | 5.6 ± 4.56 |
| Chronic | 4.0 ± 3.54 | 4.6 ± 3.29 | 4.4 ± 3.78 |

TABLE 16
Subscale 6 - Social Avoidance

| | Placebo | Low Dose | High Dose |
|---|---|---|---|
| ABC_FXS_Subscale_6 | (mean ± SD) | (mean ± SD) | (mean ± SD) |
| Baseline | 0.8 ± 1.30 | 2.0 ± 1.87 | 1.2 ± 1.79 |
| Chronic | 0 ± 0.0 | 2.4 ± 1.82 | 0.6 ± 0.89 |

In sum, results obtained from this study show that undetectable peripheral FMRP is a predictor of response. Further, low dose AZD7325 (e.g., 5 mg BID) was found to be superior over high dose (e.g., 15 mg BID) in terms of adverse effects and signal (treatment efficacy). Moreover, gamma EEG was found to be predictive of subgroup of patients who were responsive to low-dose AZD7325 treatment.

### Example 2: AZD7325 Effect on EEG Findings

Brain cells communicate via electrical impulses and are active all the time, even a subject is asleep. Electroencephalogram (EEG) detects the electrical activity in the brain. EEG can measure alpha waves, beta waves, and gamma waves. FXS patients typically show low alpha (Alpha waves are involved in higher level, complex cognition) and elevated gamma (representing noise, excessive synaptic connectivity-brain cells over connecting). Gamma activations represent high frequency brain rhythms, which may coincide with functional brain activity and/or muscular activity.

EEG statistical analysis strategies used in this study include: (a) electrode-specific relative band power estimation; (b) applying crossover-trial modeling to validate the absence of period effect; and (c) testing the within-subject relative power change per condition (i.e., determining impact of AZD7325 on gamma power in a treated patient).

Table 17 below summarizes EEG changes in response to AZD treatment.

TABLE 17
Drug Response in EEG Relative Band Power

| | |
|---|---|
| Acute visit | Chronic visit |

a) All subjects (n = 12)

| | | |
|---|---|---|
| Placebo vs High dose | theta [symbol] beta [symbol] | delta [symbol] theta [symbol] alpha1 [symbol] beta [symbol] gamma [symbol] |
| Placebo vs Low dose | theta [symbol] beta [symbol] | theta [symbol] beta [symbol] gamma [symbol] |

b) Male subjects (n = 9)

| | | |
|---|---|---|
| Placebo vs High dose | alpha2 [symbol] beta [symbol] gamma1 [symbol] | theta [symbol] alpha1 [symbol] beta [symbol] gamma [symbol] |
| Placebo vs Low dose | alpha [symbol] beta [symbol] gamma [symbol] | theta [symbol] alpha [symbol] beta [symbol] gamma1 [symbol] |

c) FMRB undetectable subjects (n = 5)

| | | |
|---|---|---|
| Placebo vs High dose | delta [symbol] alpha2 [symbol] beta [symbol] | theta [symbol] beta [symbol] gamma [symbol] |
| Placebo vs Low dose | delta [symbol] alpha [symbol] beta [symbol] | theta [symbol] alpha2 [symbol] beta [symbol] |

Further EEG band changes observed in the FXS patients treated with placebo, low dose AZD7325, or high dose AZD7325 are provided below.

(a) Low Dose Vs Placebo—Chronic Trials in all Subjects

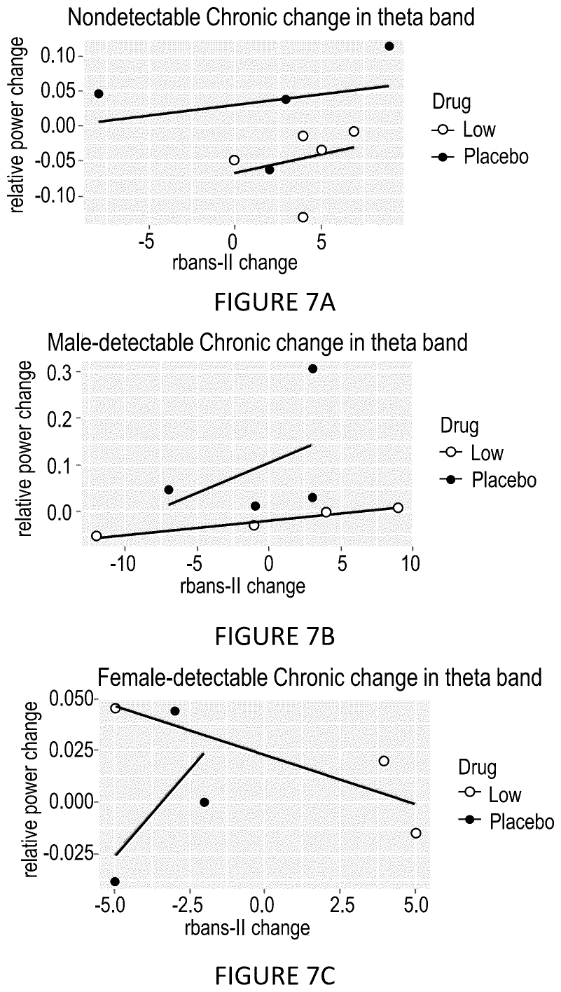

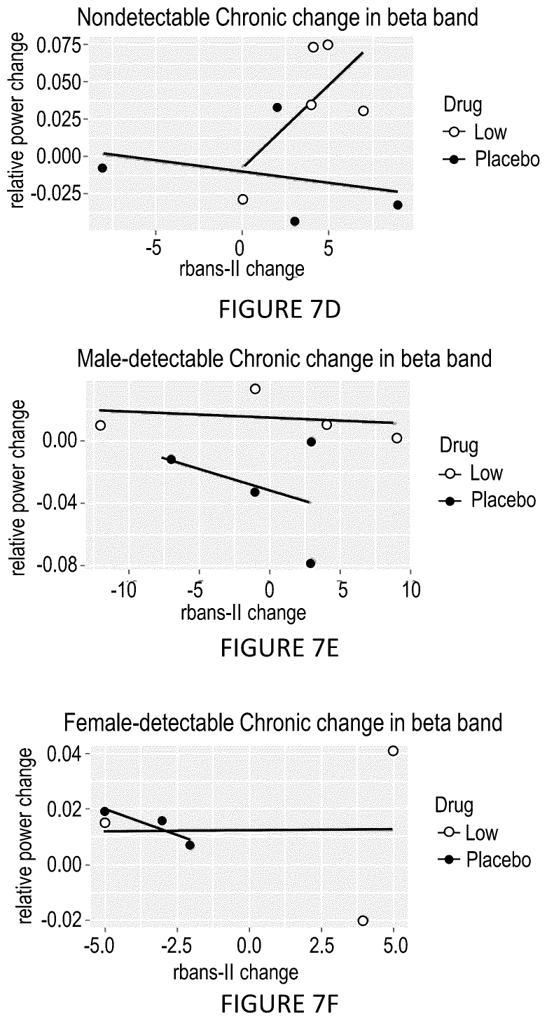

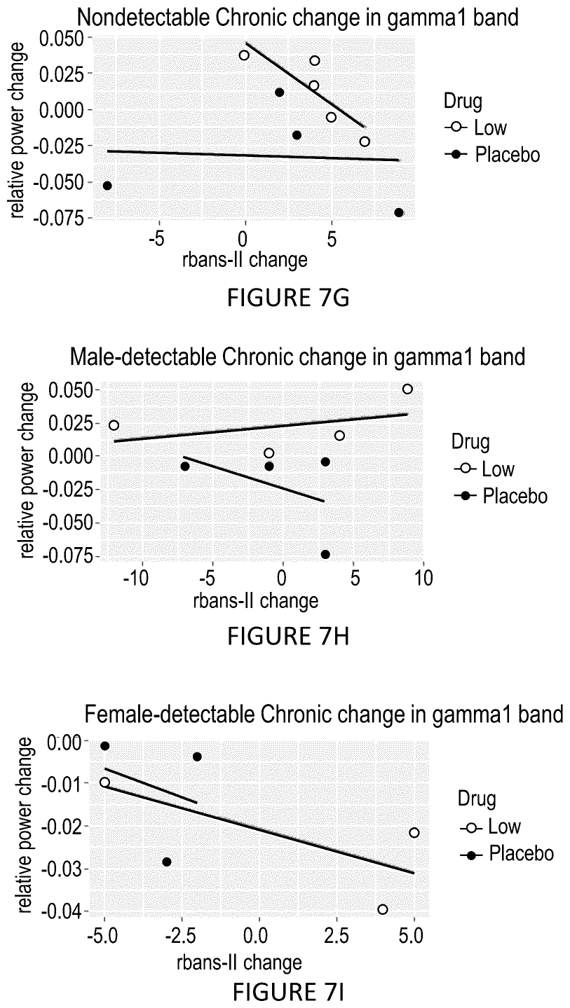

Three subgroups characterized by gender and FMRP levels were analyzed: (1) male nondetectable, (2) male detectable, and (3) female detectable. The results are shown in FIGS. 7A-7C. Chronic change correlation is summarized below:
Theta band EEG change (Y axis): approaching negative direction means more reduction. Low dose reduces theta band power in both nondetectable and male detectable groups.
RBANS_LL change (X axis): approaching positive direction means improvement in RBANS. RBANS change from nondetectable subjects are all positive trended, but upper bound doesn't exceed placebo range. RBANS from male detectable subjects are bi-directional distributed and overlap with placebo. Low dose improves RBANS growth in 2 of the three female detectable subjects.

As shown in FIGS. 7D-7F, low dose AZD7325 elevates beta power compared to placebo in the nondetectable group and in the male detectable group. Low dose AZD7325 does not effect on female EEG power.

FIGS. 7G-7I show effects on gamma power. Low dose elevates gamma 1 power (E31) compared to placebo in the nondetectable group and in the male detectable group. Low dose does not grow gamma 1 power in female subjects.

Overall, it was observed that low dose AZD7325 (see Example 1 above) reduces theta band compared to placebo and increases beta and gamma band in the parietal region.

(b) Low Dose Vs. Placebo—Chronic Trials in Male Subjects

Results from this study show that low dose AZD7325 reduces theta band and increases beta and gamma bands in the parietal in male subjects compared to placebo. Alpha bands retain consistent.

(c) Low Dose Vs. Placebo—Acute Trials

There are limited numbers of significantly effected electrodes in the theta and beta bands in terms of relative power change, comparing low dose AZD7325 to placebo in all subjects. Similarly, there are limited numbers of significantly effected electrodes in the alpha and beta bands in terms of relative power change, comparing low dose drug to placebo in male subjects. Also, there are limited numbers of significantly effected electrodes in the beta band in terms of relative power change, comparing low dose drug to placebo, in peripheral FMRP nondetectable subjects.

(d) High Dose Vs. Placebo—Chronic Trials

In all patients treated with high dose AZD7325 relative to placebo, theta band was found to decrease in the central area, while beta and gamma bands hold electrodes in the parietal and occipital areas elevated in warm colors. In male patients, theta band was found to decrease in the central area, while beta and gamma bands hold electrodes in the parietal and occipital areas elevated in warm colors. In peripheral FMRP nondetectable subjects, theta band was found to decreased in the posterior area, while beta and gamma bands hold isolated electrodes in the parietal and occipital areas elevated in warm colors.

(e) High Dose Vs. Placebo—Acute Trials

In male patients treated with high dose AZD7325 relative to placebo, central frontal power decrease was observed in the alpha2 band, beta band elevates a broad region in the central and parietal regions, and gamma 1 band has elevated electrodes in the right frontal region. In peripheral FMRP nondetectable subjects, beta band elevated broadly in the central and parietal regions, while local distant small clusters were observed in both delta and alpha2 bands.

(f) High Dose Vs. Low Dose—Chronic Trials

In all patients treated with high dose AZD7325 relative to low dose AZD7325, alpha2 band on the frontal was found to decrease, while delta, beta, gamma bands all have scattered elevated spots. In male patients treated with high dose AZD7325 relative to low dose AZD7325, alpha2 band frontal area was found to decrease in relative power, while delta band has an electrode in left central area elevated in relative band. No significant change in gamma sub-bands were observed. In peripheral FMRP nondetectable patients treated with high dose AZD7325 relative to low dose AZD7325, regions on the temporal side were found to decrease in relative power in alpha2 and beta bands, while gamma bands both have the same occipital electrode elevated.

(g) High Dose Vs. Low Dose—Acute Trials

In all patients treated with high dose AZD7325 relative to low dose AZD7325, beta band was found to elevate in multiple clusters. In male patients treated with high dose AZD7325 relative to low dose AZD7325, beta band was found to elevate in multiple clusters, and alpha2 band has a decreased power cluster in the right central region. In peripheral FMRP nondetectable patients treated with high dose AZD7325 relative to low dose AZD7325, beta band was found to elevate in multiple clusters, while left frontal has a cluster with reduced delta band power.

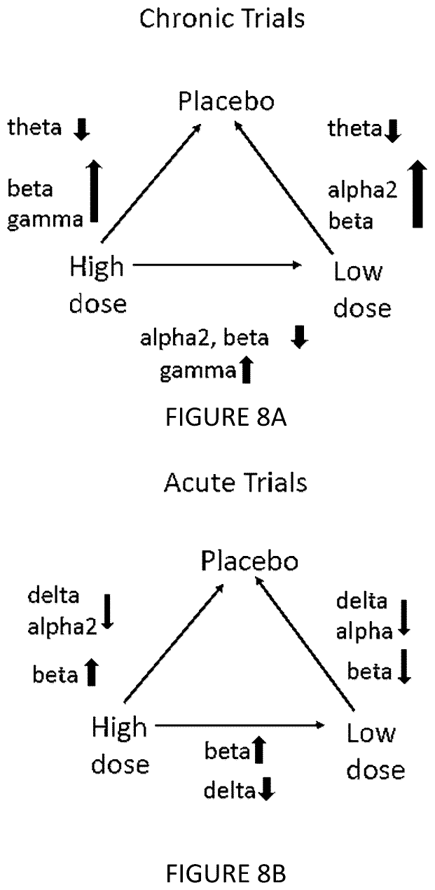

In sum, decreased gamma waves were observed in male patients having undetectable peripheral FMRP and treated with low dose AZD7325. Clinical improvement was also observed in these patients. The overall results are summarized in FIGS. 8A and 8B.

Similar results would be expected in FXS patients having a low level of peripheral FMRP, which would be indicative of poor brain distribution such that there would be essentially no biological activity of FMRP in the brain.

In sum, the results from this clinical trial indicate that peripheral FMRP, either taken alone or in combination with EEG values, can be used as a reliable biomarker for identifying FXS patients (e.g., male FXS patients) who are likely to be responsive to a low dose treatment comprising GABA_{A} stimulators such as AZD7325.

### Example 3: Luminex Assay for Measuring Levels of Peripheral FMRP

This example provides a Luminex assay that can be used for measuring levels of peripheral FMRP.

Reagent Preparation
M-PER with 150 mM NaCl (25 mL): 25 mL M-PER, in which 0.219 g of NaCl were dissolved.
Antipain (5 mg/ml): 5 mg antipain dissolved in 1 mL DMSO
Chymostatin (5 mg/mL): 5 mg chymostatin dissolved in 1 mL DMSO

Dry Blood Spot (DBS) Extraction in Microtube (Adult)

Samples extracted from DBS were prepared as follows. Prepare three 6.0-mm-diameter disks punched form the ID blood stain cards. Using clean forceps, transfer the disks into a 2 mL Spin-X Centrifuge Filter Tube (7200388, Thermo Fisher). Transfer 200 μl of M-PER (78503, Thermo Fisher) supplemented with 150 mM NaCl and stock solutions (in DMSO) of Antipain 5 mg/ml; to 10 μg/ml), Chymostatin (5 mg/ml; to 10 μg/ml); and Protease inhibitor cocktail set III—Animal-free (Calbiochem; 1:200 dilution). For 2 ml of M-PER, 5 μl of Antipain and Chymostatin and 10 μL of Protease Inhibitor Cocktail III were used. Shake tubes on Belly-dancer overnight at room temperature. Microcentrifuge tubes at 12,000×rcf at room temperature for 5 minutes. Luminex assay was run with 50 μl of liquid in five technical repeats (unless limited by number of dried blood spots).

Antibody Buffer Exchange (Innova Biosciences)

Add 100 μL of anti-FMRP 6B8 antibody (Clone: 6B8/FMRP, Biolegend) and 400 μL of 1×PBS to spin cartridge. Spin for 1 to 3 minutes in a microcentrifuge at a maximum speed of 15,000×rcf to reduce buffer volume to 100 μL. Discard the excess liquid in collection tube. Add 400 μL 1×PBS to the antibody in the spin cartridge. Spin for 1 to 3 minutes in a microcentrifuge at a recommended maximum speed of 15,000×rcf to reduce buffer volume to 100 μL. Discard the excess liquid in collection tube. Repeat steps 4-6, at least 5 times to exchange antibody buffer. Recover antibody from the spin cartridge.

Antibody Coupling (Luminex) (to MagPlex Microspheres, R33)

Remove all reagents from the refrigerator and allow them to equilibrate to room temperature for 20-30 minutes. For a 1 mL stock microsphere vial, vortex the stock microsphere vial for 10 seconds and then sonicate for 10 seconds to disperse the microspheres. Transfer 1 mL of microspheres from the stock vial into one of the microcentrifuge tubes (reaction tubes) provided in the kit.

Wash the MagPlex® Microspheres (R33) (a.k.a., beads) as follows. Place microfuge tube on magnetic separator for 2 minutes to fully pellet beads. Remove supernatant. Add 500 μL of the Activation Buffer into the reaction tube. Vortex the reaction tube for 10 seconds and then sonicate for 10 seconds to disperse the microspheres. The wash step was repeated twice.

Place the microfuge tube on magnetic separator for 2 minutes to fully pellet beads and remove supernatant. Add 400 μL of activation buffer into the reaction tube. Vortex the reaction tube for 10 seconds and then sonicate for 10 seconds to disperse the microspheres. Vortex the provided Sulfo-NHS tube for a minimum of 10 seconds. Add 50 μL of Sulfo-NHS solution to the reaction tube. Add 250 μL of Activation buffer into the 10 mg vial of EDC. Invert the EDC vial and then vortex the vial for 10 seconds to dissolve the EDC. Once opened, the EDC solution must be prepared and used quickly because it would begin to degrade once exposed to moisture in the atmosphere and in the Activation Buffer. If performing multiple reactions, be sure to prepare all of them prior to this step so that the EDC solution can be quickly added to all of the reaction tubes immediately after dissolution. The EDC must be made fresh for each coupling event and excess should be discarded.

Add 50 μL of the EDC solution into the reaction tube. Vortex the reaction tube for 15 seconds. Protect the microspheres from light and rotate on rotator for 20 minutes at a rotation speed of ˜15-30 rpm. Wash the MagPlex® Microspheres as follows. Place microfuge tube on magnetic separator for 2 minutes to fully pellet beads. Remove supernatant. Add 500 μL of the Activation Buffer into the reaction tube. Vortex the reaction tube for 10 seconds and then sonicate for 10 seconds to disperse the microspheres. The wash step was repeated for three times.

After washing, place microfuge tube on magnetic separator for 2 minutes to fully pellet beads and remove supernatant. Add 900 μL of Activation buffer and 100 μL of anti-FMRP in 1×PBS (Clone: 6B8/FMRP, Biolegend) to the reaction tube. The antibody buffer exchange must be completed prior to this step since the Tris-buffer the antibody is stored in can could interfere with the coupling reaction.

Vortex the reaction for 15 seconds. Protect the microspheres from light and rotate on rotator for 2 hours at a rotation speed of ˜15-30 rpm. Wash the MagPlex® Microspheres as follows. Place microfuge tube on magnetic separator for 2 minutes to fully pellet beads. Remove supernatant. Add 500 μL of the Wash Buffer into the reaction tube. Vortex the reaction tube for 10 seconds and then sonicate for 10 seconds to disperse the microspheres. This wash step was repeated for three times.

After washing, place the microfuge tube on magnetic separator for 2 minutes to fully pellet beads and remove supernatant. Add 1 mL of Wash Buffer into the reaction tube. The Wash Buffer can be used as a storage buffer after completing the coupling reaction. Vortex the reaction tube for 10 seconds and then sonicate for 10 seconds to disperse the microspheres. Protect from light and store at 4° C. until needed. For optimal performance, the coupled microspheres need to be blocked overnight before first use.

Luminex Assay

Buffers:

Luminex Assay Buffer (LX B) contains 1×PBS pH 7.4, 1% Bovine Serum Albumin, 0.05% Tween-20. To prepare 500 mL LX B, 300 ml of H₂O can be mixed with 50 ml of 10×PBS pH 7.4 (BP3991, Thermo Fisher), 5 g BSA, and 0.25 ml Tween-20©. Mix the solution thoroughly and bring the final volume to 500 mL. Store at 4° C. Before use in Luminex assay, bring solution to room temperature.

Work Flow

Dilute GST-SR7 standard (received from Staten Island through MTA) from −70′C stock aliquot (1:40) using mPER+antipain, chymostatin and Protease inhibitor cocktail set III—Animal-free (Calbiochem; 1:200 dilution). Generate 9-point standard by two-fold dilution from the 1st standard point. Range 70-0.275 pM. Add 50 μL of either standards or DBS extract into assay wells. Dilute anti FMRP 6B8-R33 conjugated beads to achieve 4000 beads/50 μL in Luminex Assay Buffer (LX B). Aliquot 50 μL/well of diluted 6B8-R33 beads from Step 4. Seal with aluminum foil plate seal and shake on microplate shaker and shake at 500 rpm at room temperature for 6 hours.

Wash 5 times with 200 μL per well per wash of LXB as follows. For each wash, place the plate on the magnetic plate separator for 2 mins. Invert the plate w/magnetic separator to discharge fluid. Remove plate from the magnetic separator and add wash buffer. Shake on microplate shaker at 500 rpm for 1 minute. Repeat was process for appropriate number of iterations.

After wash, add 100 μL per well 1:1000 diluted anti-FMRP rabbit polyclonal antibody (ab17722) in LXB. Mix, seal, cover, incubate at 4′C shaking overnight. (10.5 μL/10.5 mL). Wash as described above. Add 100 μL per well 1:500 diluted Goat-anti-Rabbit Phycoerythrin (PE) conjugated IgG (711-116-152, Jackson ImmunoResearch). Mix, cover, shake at room temperature for 2 hours. (21 μL/10.5 mL). Wash again as described above. Add 100 μL per well Sheath Fluid (40-50015, Luminex). Mix, cover, shake 5 min at RT. Read in LUMINEX® 200, bead region 33. For MagPlex beads, DD gating=7,500-25,000.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for identifying a Fragile X Syndrome (FXS) patient who is likely to respond to a treatment for FXS (FXS treatment), the method comprising:
(a) providing a biological sample of a FXS patient;
(b) detecting Fragile X mental retardation protein (FMRP) in the biological sample; and
(c) identifying the FXS patient for the FXS treatment, wherein the FMRP is undetectable in the biological sample or has a level no greater than a predetermined value.

2. The method of claim 1, wherein the FXS treatment comprises a GABA_{A} receptor modulator, a muscle relaxer, and/or a gene therapy, optionally wherein the FXS treatment comprises the GABA_{A} receptor modulator.

3. The method of claim 1 or claim 2, wherein the biological sample is a biofluid sample.

4. The method of claim 3, wherein the biofluid sample is a blood sample, a plasma sample, or a serum sample.

5. The method of any one of claims 1-4, wherein the FXS patient is a male human patient.

6. The method of any one of claims 1-5, wherein step (b) is performed by an immunoassay.

7. The method of claim 6, wherein the immunoassay comprises at least one antibody specific to human FMRP.

8. The method of claim 7, wherein the immunoassay comprises two antibodies specific to the human FMRP, the two antibodies binding to different epitopes of the human FMRP.

9. The method of any one of claims 1-8, further comprising performing gamma electroencephalogram (EEG) on the FXS patient to measure electrical activity of the brain.

10. The method of any one of claims 1-9, further comprising administering to the FXS patient identified in step (c) an effective amount of a GABA_{A} modulator, which optionally is a selective GABA_{A} agonist.

11. The method of claim 10, wherein the FXS patient is administered the GABA_{A} modulator, which optionally is AZD7325.

12. The method of claim 11, wherein the FXS patient is administered a low dose of the GABA_{A} modulator.

13. The method of claim 12, wherein the GABA_{A} modulator is AZD7325, which is administered to the FXS patient at 5 mg twice per day.

14. The method of any one of claims 11-13, wherein the GABA_{A} modulator is administered orally.

15. A method for treating Fragile X syndrome (FXS), comprising subjecting an FXS patient to a treatment for FXS (FXS treatment), wherein the FXS patient has undetectable peripheral Fragile X mental retardation protein (FMRP) or a level of peripheral FMRP no greater than a predetermined value.

16. The method of claim 15, wherein the FXS treatment comprises an effective amount of a GABA_{A} modulator, which optionally is a selective GABA_{A} agonist, a muscle relaxer, which optionally is baclofen, and/or a gene therapy, which optionally comprises a vector that expresses a functional FMRP polypeptide.

17. The method of claim 16, wherein the FXS treatment comprises a GABA_{A} modulator, which is AZD7325.

18. The method of claim 16, wherein the FXS patient is administered a low dose of the GABA_{A} modulator.

19. The method of claim 18, wherein the GABA_{A} modulator is AZD7325, which is administered to the FXS patient at 5 mg twice per day.

20. The method of any one of claims 16-19, wherein the GABA_{A} modulator is administered orally.

21. The method of any one of claims 15-20, further comprising performing gamma electroencephalogram (EEG) on the FXS patient before, during, and/or after the FXS treatment to monitor treatment efficacy.

22. The method of any one of claims 15-20, wherein the FXS patient is a male patient.

23. The method of any one of claims 15-22, wherein the peripheral FMRP level is measured in a biofluid sample obtained from the FXS patient.

24. The method of claim 23, wherein the biofluid sample is a blood sample, a plasma sample, or a serum sample.

25. The method of claim 23 or claim 24, wherein the peripheral FMRP level is measured by an immunoassay.

26. The method of claim 25, wherein the immunoassay comprises at least one antibody specific to human FMRP.

27. The method of claim 26, wherein the immunoassay comprises two antibodies specific to the human FMRP, the two antibodies binding to different epitopes of the human FMRP.

28. A pharmaceutical composition for use in treating Fragile X syndrome (FXS) in a FXS patient, wherein the pharmaceutical composition comprises a GABA_{A} modulator, a muscle relaxer, and/or a vector expressing a functional FMRP polypeptide, and wherein the FXS patient has undetectable peripheral Fragile X mental retardation protein (FMRP) or a level of peripheral FMRP no greater than a predetermined value.
